(19) United States
(12) Patent Application Publication
(10) Pub. No.: US 2022/0378843 A1
(43) Pub. Date: Dec. 1, 2022

(54) FIBROBLAST-BASED THERAPY FOR TREATMENT OF SCLEROSING CHOLANGITIS

(71) Applicant: FIGENE, LLC, Houston, TX (US)

(72) Inventor: Thomas ICHIM, San Diego, CA (US)

(21) Appl. No.: 17/755,051

(22) PCT Filed: Nov. 2, 2020
(86) PCT No.: PCT/US2020/058500

§ 371 Date: Apr. 20, 2022

Related U.S. Application Data
(60) Provisional application No. 62/929,250, filed on Nov. 1, 2019.

(51) Int. Cl. A61K 35/33; A61K 35/17; A61K 35/44; A61K 38/19; A61P 1/16

(52) U.S. Cl. CPC A61K 35/33; A61K 35/17; A61K 35/44; A61K 38/193; A61K 38/196; A61P 1/16

(57) ABSTRACT

In some aspects, disclosed herein are methods and compositions for treatment of sclerosing cholangitis using fibroblasts or derivatives thereof. The disclosed compositions include fibroblasts, engineered fibroblasts, exosomes obtained from fibroblasts, and conditioned media derived from fibroblasts. Methods of the present disclosure include providing fibroblasts to a subject to treat sclerosing cholangitis in the subject. Fibroblasts of the disclosure include fibroblasts capable of reducing inflammation in a subject. In certain aspects, fibroblasts are cultured with activating agents prior to therapeutic administration.

This application claims priority to U.S. Provisional Patent Application Ser. No. 62/929,250, filed Nov. 1, 2019, which is incorporated by reference herein in its entirety.

### TECHNICAL FIELD

Embodiments of the disclosure include at least the fields of cell biology, molecular biology, immunology, and medicine.

### BACKGROUND

Cholestasis, or decreased bile flow, results in a dramatic increase in liver and serum bile acid levels that may lead to acute liver toxicity, proliferation of bile ducts, and fibrosis that progresses to cirrhosis (1). Cholestasis is often divided into two categories (intrahepatic or extrahepatic) based on its etiology. Bile salts are crucial components of cholestasis and may be toxic to living cells. Early studies on the mechanisms of cholestatic liver injury strongly implicated bile acid-induced apoptosis as the major cause of hepatocellular injury. Recent work has focused on the role of both bile acids in cell signaling and sterile inflammation in the pathophysiology of cholestasis. Thus, reducing the intracellular content and cytotoxicity of bile acids (and other potentially toxic cholephilic compounds that accumulate after secretory failure) is crucial for the prevention of cholestatic liver injury.

To decrease the accumulation of unconjugated bile acids inside hepatocytes and alleviate acute cholestasis, the liver compensates through adaptive downregulation of hepatic uptake transporters and upregulation of efflux transporters. These modifications can last for a few days or weeks before liver failure or other serious liver injuries occur. To halt the process of liver fibrosis, early intervention for cholestasis is critical. These compensatory processes provide a suitable window for the treatment of cholestasis. A number of alternative drugs are currently being tested in pre-clinical studies as potential treatments for cholestatic disease, including selective modulators of nuclear receptors and signaling pathways that are thought to mediate cholestasis, however clinical efficacy has not been demonstrated to date.

Sclerosing cholangitis is a chronic cholestatic disease, characterized by inflammation, obliterative fibrosis of bile ducts, stricture formation and progressive biliary destruction leading to cirrhosis (2). Primary sclerosing cholangitis (PSC), the most common form of sclerosing cholangitis, is an idiopathic sclerosing cholangitis resulting in liver fibrosis, cirrhosis, and eventually liver failure (3). Patients with progressive PSC often require liver transplantation (4,5). In addition, immune disorders, ischemia, infections, parasites, infiltrative processes, and metastasis can cause secondary sclerosing cholangitis. However, development of effective medical therapy for sclerosing cholangitis has remained a challenge. Therefore, a new strategy to delay or prevent disease progression of sclerosing cholangitis is urgently needed.

The present disclosure satisfies a long felt need in the art for methods and compositions for treatment of sclerosing cholangitis.

### BRIEF SUMMARY

Aspects of the present disclosure are directed to fibroblasts (or components or derivatives thereof) and their use for immune modulation in the treatment of sclerosing cholangitis. In some embodiments, fibroblasts are used to induce an immunomodulated state in a subject characterized by enhanced numbers and/or activity of regulatory T cells (Tregs). Induction of such an immunomodulated state may serve to reduce pathological immunity associated with sclerosing cholangitis, thus treating the condition.

Disclosed herein, in some embodiments, are methods of treating sclerosing cholangitis in a subject comprising providing to the subject an effective amount of fibroblasts or derivatives thereof. As used herein, in certain embodiments derivatives of fibroblasts comprise conditioned media from the fibroblasts and/or exosomes from the fibroblasts. In some embodiments, the sclerosing cholangitis is primary sclerosing cholangitis. In some embodiments, the sclerosing cholangitis is secondary sclerosing cholangitis. In some embodiments, the method comprises providing an effective amount of fibroblasts to the subject, conditioned media from fibroblasts, and/or exosomes derived from fibroblasts.

In some embodiments, the fibroblasts or derivatives thereof reduce serum alkaline phosphatase levels (such as may be measured by spectrometric, spectrophotometric, or electrochemical detection techniques) in the subject by at least 35%, including reduced by 35%, 40%, 45%, 50%, and so on. In some embodiments, the fibroblasts or derivatives thereof improve an Ishak necroinflammatory grading score of the subject by at least one point. In some embodiments, the method comprises providing an effective amount of fibroblasts, conditioned media from fibroblasts, and/or exosomes from fibroblasts to a subject in need thereof, wherein the providing comprises: (a) providing a first dose of fibroblasts comprising about 100 million fibroblast cells; (b) about one, two, three, or four weeks after (a), providing a second dose of fibroblasts comprising about 100 million fibroblast cells; (c) about four, five, six, seven, or eight weeks after (a), providing a third dose of fibroblasts comprising about 100 million fibroblast cells; and (d) about four, five, six, or seven or eight weeks after (c), providing a fourth dose of fibroblasts comprising about 100 million fibroblast cells.

In some embodiments, the subject has cholestatic liver disease or is at risk for having cholestatic liver disease. In some embodiments, the subject has inflammatory bowel disease (IBD). In some embodiments, the subject does not have IBD. In some embodiments, the subject has elevated alkaline phosphatase levels (for example, compared to a standard or a general population) prior to providing the fibroblasts or derivatives thereof. In some embodiments, subsequent to the providing, the subject shows an improvement of a 5-D itch score, an Amsterdam cholestatic complaints score, or a liver stiffness transient elastography score. In some embodiments, the fibroblasts, conditioned media, and/or exosomes thereof do not cause an adverse event in the subject, wherein the adverse event is hepatoxicity, progressive multifocal leukoencephalopathy, cholangiocarcinoma, one or more complications due to portal hypertension, leucopenia, lymphopenia, colorectal cancer, infusion-related reactions, infection, acute respiratory failure, acute respiratory distress syndrome, Torsade de pointer, ventricular fibrillation, ventricular tachycardia, malignant hypertension, convulsive seizure, agranulocytosis, aplastic anemia, toxic epidermal necrolysis, Stevens-Johnson syndrome, hepatic necrosis, acute liver failure, anaphylactic shock, acute renal failure, pulmonary hypertension, pulmonary fibrosis, confirmed or suspected endotoxin shock, confirmed or suspected transmission of infectious agent by a medicinal product, neuroleptic malignant syndrome, malignant hyperthermia, spontaneous abortion, stillbirth, and/or fetal death. In some embodiments, the fibroblasts are allogenic fibroblasts, autologous fibroblasts, or xenogenic fibroblasts, or a mixture thereof. In some embodiments, the fibroblasts are derived from placenta, cord blood, peripheral blood, omentum, hair follicle, skin, bone marrow, adipose tissue, endometrium, or Wharton's Jelly.

In some embodiments, the method further comprises providing to the subject one or more additional agents, wherein the additional agent is n-acetylcysteine, ascorbic acid, alpha lipoic acid, human chorionic gonadotropin, VEGF, TNF-α, retinoic acid, alpha tocopherol, interleukin-3, G-CSF, GM-CSF, leukemia inhibitory factor, placental growth factor, angiopoietin, hydrogenated water, NGF, or a combination thereof. In some embodiments, the method further comprises providing to the subject one or more additional cell therapies, wherein the additional cell therapy is capable of suppressing liver inflammation in the subject. In some embodiments, the additional cell therapy comprises natural killer T (NKT) cells. In some embodiments, the NKT cells are activated with alpha galactosylceramide prior to providing the additional cell therapy to the subject. In some embodiments, the additional cell therapy comprises immature dendritic cells. In some embodiments, the immature dendritic cells produce more than 50 ng of interleukin-10 per 10,000,000 cells. In some embodiments, the immature dendritic cells do not express HLA II.

In some embodiments, the method further comprises providing to the subject endothelial progenitor cells. In some embodiments, the endothelial progenitor cells are derived from the subject. In some embodiments, the method further comprises mobilizing the endothelial progenitor cells in the subject. In some embodiments, mobilizing the endothelial progenitor cells comprises administration of G-CSF to the subject. In some embodiments, mobilizing the endothelial progenitor cells comprises administration of GM-CSF to the subject. In some embodiments, mobilizing the endothelial progenitor cells comprises administration of IL-3 to the subject. In some embodiments, mobilizing the endothelial progenitor cells comprises administration of TPO to the subject. In some embodiments, mobilizing the endothelial progenitor cells comprises administration of FLT3 ligand (FL) to the subject. In some embodiments, the endothelial progenitor cells are allogenic. In some embodiments, the endothelial progenitor cells are derived from placenta, cord blood, peripheral blood, omentum, hair follicle, adipose derived stromal vascular fraction, skin, bone marrow, adipose tissue, endometrium, or Wharton's Jelly.

In some embodiments, the method further comprises providing a regenerative cell to the subject. In some embodiments, the regenerative cell is a stem cell. In some embodiments, the stem cell is a hematopoietic stem cell. In some embodiments, the hematopoietic stem cell expresses CD34, CD133, c-kit, and/or thrombopoietin receptor. In some embodiments, the hematopoietic stem cell does not express CD38. In some embodiments, the hematopoietic stem cell is an autologous hematopoietic stem cell. In some embodiments, the hematopoietic stem cell is an allogenic hematopoietic stem cell. In some embodiments, the hematopoietic stem cell is a xenogenic hematopoietic stem cell. In some embodiments, the hematopoietic stem cell is derived from adipose, bone marrow, peripheral blood, mobilized peripheral blood, cord blood, or a mixture thereof. In some embodiments, the method further comprises providing to the subject mesenchymal stem cells. In some embodiments, the mesenchymal stem cellx express CD90, CD105, and/or CD73. In some embodiments, the mesenchymal stem cell does not express HLA, CD34, and/or CD14. In some embodiments, the mesenchymal stem cell is plastic adherent. In some embodiments, the mesenchymal stem cell is allogenic to the subject. In some embodiments, the mesenchymal stem cell is autologous to the subject. In some embodiments, the mesenchymal stem cell is derived from adipose, bone marrow, peripheral blood, mobilized peripheral blood, menstrual blood, fallopian tube, cord blood, or a mixture thereof. In some embodiments, the method further comprises providing to the subject an effective amount of exosomes derived from one or more stem cells, wherein the one or more stem cells comprise hematopoietic stem cells, mesenchymal stem cells, or a combination thereof. In some embodiments, the exosomes are derived from the one or more stem cells via ultracentrifugation. In some embodiments, the exosomes are derived from the one or more stem cells via chromatography. In some embodiments, the exosomes are derived from the one or more stem cells via affinity purification. In some embodiments, an outer surface of the exosomes comprises phosphatidylserine, CD9, CD19, or a tetraspanin protein. In some embodiments, the method further comprises stimulating the one or more stem cells to secrete the exosomes. In some embodiments, the stimulating comprises culturing the one or more stem cells in hypoxic conditions. In some embodiments, the hypoxic conditions comprise between 0.01% and 10% oxygen. In some embodiments, the hypoxic conditions comprise 3% oxygen. In some embodiments, the one or more stem cells are cultured in the hypoxic conditions for less than 14 days. In some embodiments, the one or more stem cells are cultured in the hypoxic conditions for about 4 days.

In some embodiments, the method further comprises culturing the fibroblasts with one or more agents and/or subjecting the fibroblasts to one or more conditions prior to providing the fibroblasts or derivatives thereof to the subject. In some embodiments, the agent is metformin. In some embodiments, the agent is oxytocin. In some embodiments, the agent is chorionic gonadotropin. In some embodiments, the agent is capable of enhancing production of an angiogenic cytokine in the fibroblasts. In some embodiments, the angiogenic cytokine is VEGF. In some embodiments, the angiogenic cytokine is FGF-1. In some embodiments, the angiogenic cytokine is FGF-2. In some embodiments, the angiogenic cytokine is IGF-1.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the FIGURES is provided for the purpose of illustration and description only and is not intended as a definition of the limits of the present disclosure.

### BRIEF DESCRIPTION OF THE DRAWINGS

FIG. 1 shows pathology score results from fibroblast treatment of cholangitis-like syndrome in rats. In the groupings of four bars, the order from left to right is as follows: control; 1 week; 2 weeks; and 4 weeks.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

As used herein, “cryopreserve,” refers to preserving cells in a cryoprotectant at a low temperature (e.g., about or less than 80° C.).

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “diluent” or “carrier” refer to a pharmaceutically acceptable (e.g., non-toxic) substance useful for the preparation of a pharmaceutical formulation. Example diluents include sterile water, bacteriostatic water for injection (BWFI), a pH buffered solution (e.g., phosphate-buffered saline), sterile saline solution, Ringer's solution, and dextrose solution.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

### II. Methods for Sclerosing Cholangitis Treatment

Disclosed herein, in some aspects, are methods for treating sclerosing cholangitis using fibroblasts and/or derivatives thereof. In some embodiments, the sclerosing cholangitis is primary sclerosing cholangitis. In some embodiments, the sclerosing cholangitis is secondary sclerosing cholangitis. In some embodiments, treating sclerosing cholangitis comprises improving the symptoms of sclerosing cholangitis in a subject. Improvement in sclerosing cholangitis symptoms may be measured by, for example, serum alkaline phosphatase levels, an Ishak necroinflammatory grading score, a 5-D itch score, an Amsterdam cholestatic complaints score, and/or a liver stiffness transient elastography score. In some embodiments, treating sclerosing cholangitis comprises preventing worsening of the symptoms of sclerosing cholangitis in a subject.

In some embodiments, methods for treating sclerosing cholangitis in a subject comprise providing to the subject an effective amount of fibroblasts. In some embodiments, methods for treating sclerosing cholangitis in a subject comprise providing to the subject an effective amount of one or more derivatives from fibroblasts (as an alternative to the fibroblasts or in addition to the fibroblasts). A “derivative” from fibroblasts describes material obtained from fibroblasts, a composition containing such material, or an agent generated from such material. For example, fibroblasts derivatives include exosomes obtained from fibroblasts, proteins (e.g., growth factors) obtained from fibroblasts, compounds (e.g., lipids, fatty acids, etc.) obtained from fibroblasts, and/or conditioned media obtained from culturing fibroblasts. In some embodiments, methods for treating sclerosing cholangitis in a subject comprise providing to the subject an effective amount of conditioned media derived from fibroblasts. In some embodiments, methods for treating sclerosing cholangitis in a subject comprise providing to the subject an effective amount of exosomes obtained from fibroblasts.

Embodiments of the present disclosure are directed to methods for treating sclerosing cholangitis that do not cause certain adverse events in a subject. For example, methods of the present disclosure comprise providing fibroblasts and/or derivatives thereof to a subject, where the fibroblasts and/or derivatives thereof do not cause an adverse event. Examples of adverse events that are avoided by the disclosed methods include hepatoxicity, progressive multifocal leukoencephalopathy, cholangiocarcinoma, one or more complications due to portal hypertension, leucopenia, lymphopenia, colorectal cancer, infusion-related reactions, infection, acute respiratory failure, acute respiratory distress syndrome, Torsade de pointer, ventricular fibrillation, ventricular tachycardia, malignant hypertension, convulsive seizure, agranulocytosis, aplastic anemia, toxic epidermal necrolysis, Stevens-Johnson syndrome, hepatic necrosis, acute liver failure, anaphylactic shock, acute renal failure, pulmonary hypertension, pulmonary fibrosis, confirmed or suspected endotoxin shock, confirmed or suspected transmission of infectious agent by a medicinal product, neuroleptic malignant syndrome, malignant hyperthermia, spontaneous abortion, stillbirth, and/or fetal death.

In some embodiments, fibroblasts are cultured with one or more agents prior to being provided to a subject. In some embodiments, fibroblasts are cultured with one or more agents capable of enhancing production of one or more angiogenic cytokines in the fibroblasts. In some embodiments, fibroblasts are cultured with metformin, oxytocin, and/or chorionic gonadotropin.

In some embodiments, fibroblasts, conditioned media derived from fibroblasts, and/or exosomes derived from fibroblasts are provided to a subject together with one or more additional components. In some embodiments, fibroblasts are provided to a subject prior to providing one or more additional components. In some embodiments, fibroblasts are provided to a subject after providing one or more additional components. In some embodiments, fibroblasts are provided to a subject substantially simultaneously with providing one or more additional components. The one or more additional components may include, for example, an additional cell therapy, endothelial progenitor cells, a regenerative cell (e.g., stem cell), a mesenchymal stem cell, exosomes derived from stem cells, or a combination thereof.

In some embodiments, fibroblasts or derivatives thereof are provided to a subject in combination with one or more additional agents selected from n-acetylcysteine, ascorbic acid, alpha lipoic acid, human chorionic gonadotropin, VEGF, TNF-α, retinoic acid, alpha tocopherol, interleukin-3, G-CSF, GM-CSF, leukemia inhibitory factor, placental growth factor, angiopoietin, hydrogenated water, and NGF.

In some embodiments, fibroblasts or derivatives thereof are provided to a subject in combination with an additional cell therapy. An additional cell therapy may be a therapy capable of suppressing liver inflammation in the subject. In some embodiments, an additional cell therapy comprises natural killer T (NKT) cells. In some embodiments, the NKT cells are activated with alpha galactosylceramide prior to being provided to the subject. In some embodiments, the additional cell therapy comprise immature dendritic cells. In some embodiments, the immature dendritic cells produce greater than 50 ng of IL-10 per 10,000,000 cells. In some embodiments, the immature dendritic cells do not express HLA II.

In some embodiments, fibroblasts are provided to a subject in combination with endothelial progenitor cells (EPCs). The EPCs may be allogenic or autologous. EPCs may be derived from one or more sources, including peripheral blood, mobilized peripheral blood, bone marrow, adipose derived stromal vascular fraction, cord blood, Wharton's jelly, and placenta. In some embodiments, EPCs are derived from peripheral blood. In some embodiments, EPCs are mobilized from a subject prior to being derived from the subject. EPCs may be mobilized by, for example, administering G-CSF, GM-CSF, IL-3, TPO, FLT3 ligand (FL), or a combination thereof.

In some embodiments, fibroblasts or derivatives thereof are provided to a subject in combination with a one or more regenerative cells. A regenerative cell may be a stem cell. In some embodiments, fibroblasts are provided to a subject in combination with one or more hematopoietic stem cells. The hematopoietic stem cells may be autologous, allogenic, or xenogenic. Hematopoietic stem cells may be derived from one or more sources including adipose, bone marrow, peripheral blood, mobilized peripheral blood, and cord blood. In some embodiments, hematopoietic stem cells are derived from bone marrow. In some embodiments, the hematopoietic stem cells express CD34, CD133, and/or c-kit. In some embodiments, the hematopoietic stem cells do not express CD38 and/or thrombopoietin. In some embodiments, fibroblasts are provided to a subject in combination with one or more mesenchymal stem cells. The mesenchymal stem cells may be autologous, allogenic, or xenogenic. Mesenchymal stem cells may be derived from one or more sources including adipose, bone marrow, peripheral blood, mobilized peripheral blood, menstrual blood, fallopian tube, or cord blood. In some embodiments, the mesenchymal stem cells express CD90, CD105, and/or CD73. In some embodiments, the mesenchymal stem cells do not express HLA, CD34, or CD14.

In some embodiments, fibroblasts and/or derivatives thereof are provided to a subject in combination with a one or more anti-inflammatory agents. An anti-inflammatory agent may be any agent capable of reducing or preventing an immune response in a subject. In some embodiments, the one or more anti-inflammatory agents comprise interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric .acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, 3-stearyloxy-glycyrrhetinic acid, or a combination thereof.

It is contemplated that methods and compositions comprising fibroblasts may comprise, in addition or in place of the fibroblasts, conditioned media from fibroblasts and/or exosomes derived from fibroblasts. As such, where methods discussed describe embodiments comprising the use of fibroblasts, also contemplated are embodiments where conditioned media and/or exosomes derived from fibroblasts are substituted for the fibroblasts. One or more of fibroblasts, conditioned media from fibroblasts, and exosomes derived from fibroblasts may be used alternatively or in combination in the disclosed methods and compositions.

### III. Immune Modulation

Embodiments of the disclosure are directed to compositions comprising fibroblasts (or derivatives) thereof and methods for use in immune modulation. Abnormal immunity has been reported for sclerosing cholangitis. Numbers of regulatory T cells (Tregs) in peripheral blood of sclerosing cholangitis subjects are significantly decreased relative to healthy subjects (24). The role of regulatory T cells (Tregs) is universally associated with tolerance in conditions of natural tolerance such as in pregnancy (6,7), transplantation tolerance (8-12), and ocular tolerance (13-22). The functional relevance of Treg cells to preservation and/or initiation of tolerance is observed in conditions where administration of Tregs prevents pathology, such as in spontaneous abortion (23). Accordingly, in some embodiments, provided are methods comprising using Tregs (e.g., enhancement of Treg number or activity, administration of Tregs, etc.) for the treatment of sclerosing cholangitis. In some embodiments, fibroblasts are provided to a subject to increase number and/or activity of regulatory T cells (Tregs) in the subject. In some embodiments, increasing the number or activity of Tregs in a subject serves to reduce pathological immunity associated with sclerosing cholangitis.

In some embodiments, fibroblasts are used to accelerate regeneration of certain regions of the liver in a subject with sclerosing cholangitis. In some embodiments, fibroblasts are used to support regeneration of hepatic function. In some embodiments, fibroblasts are provided in an amount and manner sufficient to stimulate hepatocyte proliferation in a subject while also reducing chronic inflammation.

### IV. Fibroblasts and Cultured Cells

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells. In one example, fibroblasts are isolated and provided to a subject together with one or more endothelial progenitor cells. In some embodiments, disclosed herein are fibroblasts capable of stimulating tissue regeneration, immune modulation, and/or angiogenesis.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media. Determination of expression of a cell surface marker can be measured (e.g., via flow cytometry) using cutoff values as obtained from a negative control sample (e.g., a sample known not to express the surface marker of interest) and/or an isotype control. In some embodiments, a cell is determined not to express a cell surface marker if the results are about the same as those obtained for the negative control sample and/or isotype control.

Fibroblasts may be expanded and utilized by administration themselves, or may be cultured in a growth media in order to obtain conditioned media. The term Growth Medium generally refers to a medium sufficient for the culturing of fibroblasts. In particular, one presently preferred medium for the culturing of the cells of the invention herein comprises Dulbecco's Modified Essential Media (DMEM). Particularly preferred is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, Hyclone™, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present invention, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. In specific cases, these cell numbers are produced within 80, 70, or 60 days or less. In one embodiment, fibroblast cells used are isolated and expanded, and possess one or more markers selected from the group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be obtained from a source selected from the group consisting of: dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and a combination thereof. In some embodiments, fibroblasts are dermal fibroblasts.

In some embodiments, disclosed are cells that require no exogenous growth factors, except as are available in supplemental serum provided with the Growth Medium. Also provided herein are methods of deriving umbilical cells capable of expansion in the absence of particular growth factors. Such methods may require that the particular growth factors (for which the cells have no requirement) be absent in the culture medium in which the cells are ultimately resuspended and grown. In this sense, the method is selective for those cells capable of division in the absence of the particular growth factors. In some embodiments, cells are capable of growth and expansion in chemically-defined growth media with no serum added. In such cases, the cells may require certain growth factors, which can be added to the medium to support and sustain the cells. Example factors that may be added for growth on serum-free media include one or more of FGF, EGF, IGF, and PDGF. In some embodiments, two, three, or all four of the factors are add to serum free or chemically defined media. In other embodiments, leukemia inhibitory factor (LIF) is added to serum-free medium to support or improve growth of the cells.

In some embodiments, fibroblasts are transfected with one or more angiogenic genes. An “angiogenic gene” describes a gene encoding for a protein or polypeptide capable of stimulating or enhancing angiogenesis in a culture system, tissue, or organism. Examples of angiogenic genes that may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-βreceptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. In some embodiments, angiogenic genes are angiogenic cytokines. In some embodiments, fibroblasts express one or more of VEGF, FGF-1, FGF-2, and IFG-1. Fibroblasts transfected with one or more angiogenic factors may be used in the disclosed methods of treatment of sclerosing cholangitis.

In some embodiments, fibroblasts and/or other cells of the present disclosure (e.g., endothelial progenitor cells, mesenchymal stem cells, hematopoietic stem cells, etc.) are treated with (e.g., cultured with) metformin prior to use or administration to a subject. Treatment with metformin may enhance therapeutic efficacy. In some embodiments, fibroblasts are preconditioned with metformin to enhance their angiogenic potential prior to therapeutic use. Angiogenic potential may be assessed by measurement of the production of cytokines such as VEGF, EGF, IGF, FGF-1, FGF-2, and PGF.

### V. Obtaining and Processing Fibroblasts

Fibroblasts may be obtained from various sources using methods known in the art and those disclosed herein. Example methods for deriving fibroblasts from skin tissue are provided.

In some embodiments, skin tissue (dermis and epidermis layers) are biopsied from a subject's post-auricular area. The starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±0.2° C. for one hour. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask (e.g., T-175, T-75, T-150, T-185 or T-225) for initiation of cell growth and expansion. Cells are incubated at 37.0±0.2° C. with 5.0.±1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the flask, the cells are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37.0±0.2° C. with 5.0±1.0% CO₂ and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is about 95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37.0±0.2° C. with 5.0±1.0% CO₂ and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

In some embodiments, when large numbers of cells are required after receiving cell count results from the primary 10 CS harvest, an additional passage into multiple cell stacks (up to four 10 CS) is performed. For additional passaging, cells from the primary harvest are added to a 2 L media bottle containing fresh Complete Growth Media. Resuspended cells are added to multiple cell stacks and incubated at 37.0±0.2° C. with 5.0±1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above. The method decreases or eliminates immunogenic proteins by avoiding their introduction from animal-sourced reagents. To reduce process residuals, cells are cryopreserved in protein-free freeze media, then thawed and washed prior to prepping the final injection to further reduce remaining residuals. If additional Drug Substance is needed after the harvest and cryopreservation of cells from additional passaging is complete, aliquots of frozen Drug Substance—Cryovial are thawed and used to seed 5 CS or 10 CS culture vessels. Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest.

At the completion of culture expansion, the cells are harvested and washed, then formulated to contain 1.0-2.7×10⁷ cells/mL, with a target of 2.2×10⁷ cells/mL. Alternatively, the target can be adjusted within the formulation range to accommodate different indication doses. The drug substance consists of a population of viable, autologous human fibroblast cells suspended in a cryopreservation medium consisting of Iscove's Modified Dulbecco's Medium (IMDM) and Profreeze™-CDM (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. In addition to cell count and viability, purity/identity of the Drug Substance is performed and must confirm the suspension contains 98% or more fibroblasts. The usual cell contaminants include keratinocytes. The purity/identify assay employs fluorescent-tagged antibodies against CD90 and CD104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin (34 chain, is a 205 kDa transmembrane glycoprotein which associates with integrin α6 chain (CD49f) to form the α6/β4 complex. This complex has been shown to act as a molecular marker for keratinocyte cells.

Antibodies to CD104 protein bind to 100% of human keratinocyte cells. Cell count and viability is determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead. The method was custom developed specifically for use in determining purity/identity of autologous cultured fibroblasts. Alternatively, cells can be passaged from either the T-175 flask (or alternatives) or the T-500 flask (or alternatives) into a spinner flask containing microcarriers as the cell growth surface. Microcarriers are small bead-like structures that are used as a growth surface for anchorage dependent cells in suspension culture. They are designed to produce large cell yields in small volumes. In this apparatus, a volume of Complete Growth Media ranging from 50 mL-300 mL is added to a 500 mL, 1 L or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37.0±0.2° C. with 5.0±1.0% CO₂ incubator to allow for adherence of cells to the carriers. After the attachment period, the speed of the spin plate is increased (30-120 RPM). Cells are fed with fresh Complete Growth Media every one to five days, or when media appears spent by color change. Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsiziation and reseeding. Alternatively, if enough cells are produced from the initial T-175 or T-500 flask, the cells can be directly seeded into the scale-up amount of microcarriers. After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA. Microcarriers used within the disposable spinner flask may be made from poly blend such as BioNOC II™ (Cesco Bioengineering, distributed by Bellco Biotechnology, Vineland, N.J.) and FibraCel™ (New Brunswick Scientific, Edison, N.J.), gelatin, such as Cultispher-G (Percell Biolytica, Astrop, Sweden), cellulose, such as Cytopore™ (GE Healthcare, Piscataway, N.J.) or coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah).

In some embodiments, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. In some embodiments, serine protease are used in a sequence following the use of other enzymes in order to degrade the other enzymes being used. Serine proteases may be inhibited with alpha 2 microglobulin in serum and therefore the medium used for digestion may be serum-free. EDTA and DNase are commonly used and may improve yields or efficiencies. Example methods involve enzymatic treatment with collagenase and dispase, or collagenase, dispase, and hyaluronidase, and such methods are provided wherein a mixture of collagenase and the neutral protease dispase are used in the dissociating step. Also disclosed are those methods which employ digestion in the presence of at least one collagenase from Clostridium histolyticum, and either of the protease activities, dispase and thermolysin. Also disclosed are methods employing digestion with both collagenase and dispase enzyme activities. Also disclosed are methods which include digestion with a hyaluronidase activity in addition to collagenase and dispase activities. The skilled artisan will appreciate that many such enzyme treatments are known in the art for isolating cells from various tissue sources. For example, the Liberase™ blends (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.0° C. during the enzyme treatment of the dissociation step. Diluting the digest may also improve yields of cells as cells may be trapped within a viscous digest.

In some embodiments, the use of enzyme activities is not used or required for obtaining fibroblasts or other cells. In some embodiments, mechanical separation is used alone to isolate fibroblasts. Cells can be resuspended after the tissue is dissociated into any culture medium as discussed herein. Cells may be resuspended following a centrifugation step to separate out the cells from tissue or other debris. Resuspension may involve mechanical methods of resuspending, or simply the addition of culture medium to the cells.

### VI. Exosomes

Exosomes, as used herein, describe nanovesicles released from a variety of cells. Exosomes may be derived from large multivesicular endosomes and secreted into the extracellular milieu. Exosomes, also referred to as “microparticles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/ml and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts or medium conditioned by the fibroblasts for the purpose of for example treating or preventing a disease (e.g., stroke). The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins. In some embodiments, a microparticle of the present disclosure comprises phosphatidylserine, CD9, CD19, and/or a tetraspanin protein on its surface. The microparticles may comprise mRNA and/or microRNA. The microparticle may be used for any of the therapeutic purposes that the fibroblasts or fibroblast conditioned media may be used for.

In one embodiment, fibroblast exosomes, or microparticles may be produced by culturing fibroblasts in a medium to condition them. The fibroblasts may be derived from human umbilical tissue derived cells, or other tissues possessing or associated with regenerative features, which possess markers selected from a group consisting of CD90, CD73, CD105, and a combination thereof. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or β-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6×40 mm or a TSK gel G4000 SWXL, 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rh of microparticles in this peak is about 45-55 nm.

Exosomes may be purified using a variety of means known in the art. The fibroblast cell-derived microparticles of the present disclosure can be recovered by carrying out expansion culturing of fibroblast cells in conditioned medium to subconfluency (or confluency), replacing the medium with fresh conditioned medium, culturing for normally 1 to 5 days (e.g., 1 day, 2 to 3 days, or 3 to 4 days) and recovering the microparticles from the culture supernatant. Examples of methods used to recover the fibroblast cell-derived microparticles of the present disclosure include ultracentrifugation, density gradient centrifugation and the use of various types of exosome separation kits (such as the formation of a pellet by centrifugation, immunoprecipitation, purification with magnetic beads, fractionation according to particle size or column adsorption). In some embodiments, methods used to recover fibroblast cell-derived microparticles of the present disclosure includes subjecting a culture supernatant of fibroblast cells to ultracentrifugation for 0.5 hours to 2 hours at about 50,000 G to 150,000 G. The method can also include centrifuging the culture supernatant of the fibroblast cells to 0.1 hours to 2 hours at about 100 G to 20,000 G prior to carrying out ultracentrifugation. The fibroblast cell-derived microparticles of the present disclosure can be stored for about 1 week at 4° C., for about 1 month at −20° C. or for about 6 months at −80° C., provided it is stored dissolved in a solution such as PBS, and can be stored for about 3 years at 4° C. provided it has been freeze-dried. In some embodiments, exosomes are purified using ultracentrifugation. In some embodiments, exosomes are purified using chromatography. In some embodiments, exosomes are purified using affinity purification.

Exosomes of the present disclosure may be derived from one or more cellular sources. In some embodiments, exosomes are derived from fibroblasts. In some embodiments, exosomes are derived from regenerative fibroblasts. In some embodiments, exosomes are derived from stem cells (e.g., hematopoietic stem cells, mesenchymal stem cells, etc.). In some embodiments, exosomes are derived by isolating conditioned media from a cell culture. Exosomes, provided alone or as a component of conditioned media, may be used in any of the disclosed therapeutic methods or compositions.

The disclosed methods may comprise administration of exosomes to a subject. Compositions comprising exosomes may comprise about, at least, or at most 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 445, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 nanograms (ng), micrograms (mcg), milligrams (mg), or grams of exosomes, or any range derivable therein. Exosomes may be administered at a dose based on a subject's weight, expressed as ng/kg, mg/kg, or g/kg.

### VII. Administration of Therapeutic Compositions

The therapy provided herein may comprise administration of a therapeutic agents alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Compositions (e.g., comprising fibroblasts or derivatives thereof) may be administered using a “dosing regimen” or “therapeutic regimen”, which describe one or more unit doses administered to a subject. In some embodiments, a dosing regimen comprises a plurality of doses each separated by a time period of the same length. In some embodiments, a dosing regimen comprises a plurality of doses each separated by a time period of varying lengths. In some embodiments, a composition is administered continuously over a predetermined amount of time. In some embodiments, a composition is administered once a day (QD) or twice a day (BID).

In some embodiments, fibroblasts are administered to a subject according to a dosing regimen comprising one or multiple doses, and they may be administered locally or systemically. In specific cases, the cells are administered as an initial dose, a second subsequent dose, optionally a third subsequent dose, optionally a fourth subsequent dose, and so on. In some embodiments, an initial dose comprises between 50 million and 200 million fibroblast cells, or any range derivable therein. In some embodiments, an initial dose comprises about 100 million fibroblast cells. In some embodiments, an initial dose is provided via intravenous infusion. In some embodiments, a second subsequent dose comprises between 50 million and 200 million fibroblast cells, or any range derivable therein. In some embodiments, a second subsequent dose comprises about 100 million fibroblast cells. In some embodiments, a second subsequent dose is provided via intravenous infusion. In some embodiments, a second subsequent dose is provided between one week and three weeks after an initial dose, or any range derivable therein. In some embodiments, a second subsequent dose is provided about two weeks after an initial dose. In some embodiments, a third subsequent dose comprises between 50 million and 200 million fibroblast cells, or any range derivable therein. In some embodiments, a third subsequent dose comprises about 100 million fibroblast cells. In some embodiments, a third subsequent dose is provided via intravenous infusion. In some embodiments, a third subsequent dose is provided between three weeks and nine weeks after a second subsequent dose, or any range derivable therein. In some embodiments, a third subsequent dose is provided about six weeks after a second subsequent dose. In some embodiments, a fourth subsequent dose comprises between 50 million and 200 million fibroblast cells, or any range derivable therein. In some embodiments, a fourth subsequent dose comprises about 100 million fibroblast cells. In some embodiments, a fourth subsequent dose is provided via intravenous infusion. In some embodiments, a fourth subsequent dose is provided between three weeks and nine weeks after a third subsequent dose, or any range derivable therein. In some embodiments, a fourth subsequent dose is provided about four weeks after a third subsequent dose. In some embodiments, a fourth subsequent dose is provided about eight weeks after a third subsequent dose.

### VIII. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means. Any compound or composition or protein, etc., described herein may be provided in a kit.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### In Vivo Treatment of Cholangitis-Like Syndrome Using Fibroblasts

Human dermal fibroblasts where obtained from American Type Culture Collection (ATCC) and cultured in the presence of 1 μM oxytocin for 48 hours.

Six-week-old male Sprague-Dawley rats were housed 3 rats per cage in a temperature-controlled room (24° C.) on a 12 hr/12 hr light/dark cycle. All rats had ad libitum access to standard pellets throughout the study including the 5-day acclimatization period. In order to induce a sclerosing cholangitis-like syndrome, a 1.79-mm-diameter tube was inserted into the stomach from the mouth. Cholangitis was induced via the intragastric administration of 100 mg/kg alpha-naphthylisothiocyanate (ANIT) (Sigma-Aldrich, St. Louis, Mo., USA) in 500 μL of olive oil (Wako Pure Chemical Industries, Osaka, Japan) twice weekly for 4 weeks. In the control group, rats were administrated olive oil alone.

In the ANIT+ fibroblast group (N=10), one million fibroblasts suspended in 200 μL of PBS were intravenously injected through the tail vein on days 15 and 22 after administration of ANIT.

Rats were sacrificed on at either day 0, 1 week, 2 weeks, or 4 weeks. The abdomens of rats were opened under anesthesia with 50 mg/kg intraperitoneal pentobarbital. In each animal, the left lobe of the liver was removed, fixed in 40 g/L formaldehyde saline, embedded in paraffin, cut into 5-μm sections, and stained with hematoxylin and eosin (H&E; Wako Pure Chemical Industries) and Sirius Red (Wako Pure Chemical Industries. Biliary hyperplasia, fibroblast proliferation, and biliary neutrophilic infiltration were scored as described previously as follows: 0, no evidence of abnormality; 1, minimal; 2, mild; 3, moderate; and 4, severe. The number of necrotic lesions in H&E-stained sections was quantitatively measured as described previously. To quantify collagen deposits, 10 random fields (×100) of Sirius Red-stained sections from each rat were photographed, and red-stained areas were measured using a digital image analyzer.

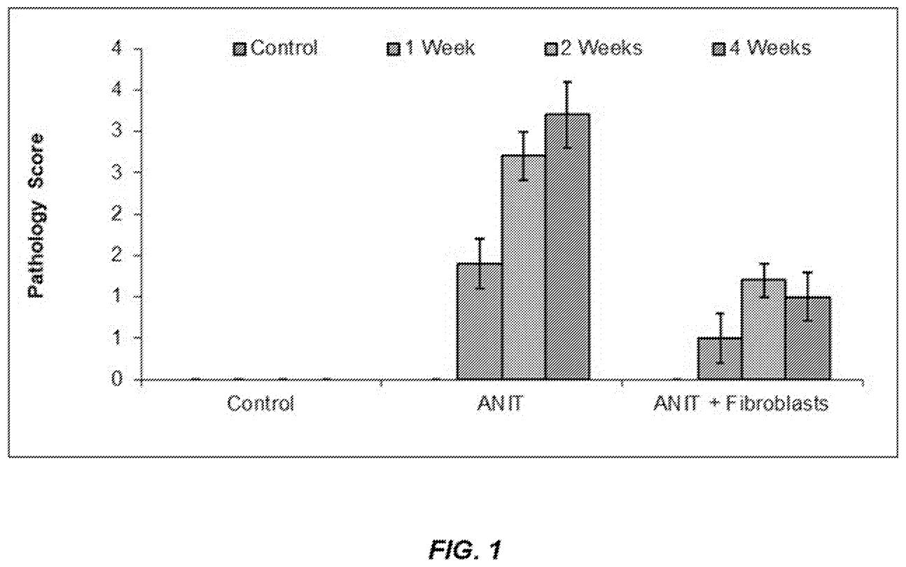

A significant reduction in pathology score was observed in the treated rats, as shown in FIG. 1.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
1. Hay, J. E. (1998) Liver transplantation for primary biliary cirrhosis and primary sclerosing cholangitis: does medical treatment alter timing and selection? Liver Transpl Surg 4, S9-17
2. Abdalian, R., and Heathcote, E. J. (2006) Sclerosing cholangitis: a focus on secondary causes. Hepatology 44, 1063-1074
3. Lazaridis, K. N., and LaRusso, N. F. (2016) Primary Sclerosing Cholangitis. N Engl J Med 375, 1161-1170
4. Geonzon-Gonzales, M. R. (2007) Primary sclerosing cholangitis. Gastroenterol Nurs 30, 102-105; quiz 105-107
5. Michaels, A., and Levy, C. (2008) The medical management of primary sclerosing cholangitis. Medscape J Med 10, 61
6. Aluvihare, V. R., Kallikourdis, M., and Betz, A. G. (2004) Regulatory T cells mediate maternal tolerance to the fetus. Nat Immunol 5, 266-271
7. Somerset, D. A., Zheng, Y., Kilby, M. D., Sansom, D. M., and Drayson, M. T. (2004) Normal human pregnancy is associated with an elevation in the immune suppressive CD25+CD4+ regulatory T-cell subset. Immunology 112, 38-43
8. Campos-Mora, M., Contreras-Kallens, P., Galvez-Jiron, F., Rojas, M., Rojas, C., Refisch, A., Cerda, O., and Pino-Lagos, K. (2019) CD4+Foxp3+T Regulatory Cells Promote Transplantation Tolerance by Modulating Effector CD4+ T Cells in a Neuropilin-1-Dependent Manner. Front Immunol 10, 882
9. Lalfer, M., Chappert, P., Carpentier, M., Urbain, D., Davoust, J. M., and Gross, D. A. (2019) Foxp3(+) Regulatory and Conventional CD4(+) T Cells Display Similarly High Frequencies of Alloantigen-Reactive Cells. Front Immunol 10, 521
10. Wang, K., Song, Z. L., Wu, B., Zhou, C. L., Liu, W., and Gao, W. (2019) Different phenotypes of CD4(+)CD25(+)Foxp3(+) regulatory T cells in recipients post liver transplantation. Int Immunopharmacol 69, 194-201
11. Herrera-Gomez, F., Del Aguila, W., Tejero-Pedregosa, A., Adler, M., Padilla-Berdugo, R., Maurtua-Briseno-Meiggs, A., Pascual, J., Pascual, M., San Segundo, D., Heidt, S., Alvarez, F. J., Ochoa-Sangrador, C., and Lambert, C. (2018) The number of FoxP3 regulatory T cells in the circulation may be a predictive biomarker for kidney transplant recipients: A multistage systematic review. Int Immunopharmacol 65, 483-492
12. Leung, C. S., Yang, K. Y., Li, X., Chan, V. W., Ku, M., Waldmann, H., Hori, S., Tsang, J. C. H., Lo, Y. M. D., and Lui, K. O. (2018) Single-cell transcriptomics reveal that PD-1 mediates immune tolerance by regulating proliferation of regulatory T cells. Genome Med 10, 71
13. Zhu, X., Yang, P., Zhou, H., Li, B., Huang, X., Meng, Q., Wang, L., and Kijlstra, A. (2007) CD4+CD25+Tregs express an increased LAG-3 and CTLA-4 in anterior chamber-associated immune deviation. Graefes Arch Clin Exp Ophthalmol 245, 1549-1557
14. Zhang, H., Yang, P., Zhou, H., Meng, Q., and Huang, X. (2008) Involvement of Foxp3-expressing CD4+CD25+ regulatory T cells in the development of tolerance induced by transforming growth factor-beta2-treated antigen-presenting cells. Immunology 124, 304-314
15. He, H., Yang, P., Jiang, L., Zhang, J., Zhao, C., Chen, L., Lin, X., Zhou, H., and Kijlstra, A. (2008) Upregulation of CD94 on CD8+T cells in anterior chamber-associated immune deviation. BMC Immunol 9, 53
16. Saban, D. R., Cornelius, J., Masli, S., Schwartzkopff, J., Doyle, M., Chauhan, S. K., Peck, A. B., and Grant, M. B. (2008) The role of ACAID and CD4+CD25+FOXP3+ regulatory T cells on CTL function against MHC alloantigens. Mol Vis 14, 2435-2442
17. Hori, J., Taniguchi, H., Wang, M., Oshima, M., and Azuma, M. (2010) GITR ligand-mediated local expansion of regulatory T cells and immune privilege of corneal allografts. Invest Ophthalmol Vis Sci 51, 6556-6565
18. Ji, S. X., Yin, X. L., and Yang, P. Z. (2011) Effect of CD4(+)CD25(+) regulatory T cells in the development of anterior chamber-associated immune deviation. Int J Ophthalmol 4, 19-25
19. Zhang, Y., Zhang, M., Zhao, S., Li, X., Jia, Z., Zhang, L., Han, Z. C., and Zhang, X. (2013) Effects of human umbilical cord-derived mesenchymal stem cells on anterior chamber-associated immune deviation. Int Immunopharmacol 15, 114-120
20. Yan, F., Cai, L., Hui, Y., Chen, S., Meng, H., and Huang, Z. (2014) Tolerogenic dendritic cells suppress murine corneal allograft rejection by modulating CD28/CTLA-4 expression on regulatory T cells. Cell Biol Int 38, 835-848
21. Wang, T., Shi, W., Fan, T., Wan, X., Chen, Y. H., and Ruan, Q. (2016) c-Rel is Required for the Induction of pTregs in the Eye but Not in the Gut Mucosa. Immunol Invest 45, 776-786
22. Kunishige, T., Taniguchi, H., Terada, M., Akiba, H., Yagita, H., Abe, R., and Hori, J. (2016) Protective Role of ICOS and ICOS Ligand in Corneal Transplantation and in Maintenance of Immune Privilege. Invest Ophthalmol Vis Sci 57, 6815-6823
23. Zenclussen, A. C., Gerlof, K., Zenclussen, M. L., Sollwedel, A., Bertoja, A. Z., Ritter, T., Kotsch, K., Leber, J., and Volk, H. D. (2005) Abnormal T-cell reactivity against paternal antigens in spontaneous abortion: adoptive transfer of pregnancy-induced CD4+CD25+T regulatory cells prevents fetal rejection in a murine abortion model. Am J Pathol 166, 811-822
24. Sebode, M., Peiseler, M., Franke, B., Schwinge, D., Schoknecht, T., Wortmann, F., Quaas, A., Petersen, B. S., Ellinghaus, E., Baron, U., Olek, S., Wiegard, C., Weiler-Normann, C., Lohse, A. W., Herkel, J., and Schramm, C. (2014) Reduced FOXP3(+) regulatory T cells in patients with primary sclerosing cholangitis are associated with IL2RA gene polymorphisms. J Hepatol 60, 1010-1016

What is claimed is:

1. A method of treating sclerosing cholangitis in a subject comprising providing to the subject an effective amount of fibroblasts or derivatives thereof.

2. The method of claim 1, wherein the sclerosing cholangitis is primary sclerosing cholangitis.

3. The method of claim 1, wherein the sclerosing cholangitis is secondary sclerosing cholangitis.

4. The method of any of claims 1-3, wherein the method comprises providing an effective amount of fibroblasts to the subject.

5. The method of any of claims 1-3, wherein the method comprises providing an effective amount of conditioned media from fibroblasts to the subject.

6. The method of any of claims 1-3, wherein the method comprises providing an effective amount of exosomes derived from fibroblasts to the subject.

7. The method of any of claims 1-6, wherein the fibroblasts or derivatives thereof reduce serum alkaline phosphatase levels in the subject by at least 35%.

8. The method of any of claims 1-7, wherein the fibroblasts or derivatives thereof improve an Ishak necroinflammatory grading score of the subject by at least one point.

9. The method of any of claims 1-8, wherein the method comprises providing an effective amount of fibroblasts to the subject, wherein the providing comprises:
(a) providing a first dose of fibroblasts comprising about 100 million fibroblast cells;
(b) about two weeks after (a), providing a second dose of fibroblasts comprising about 100 million fibroblast cells;
(c) about six weeks after (a), providing a third dose of fibroblasts comprising about 100 million fibroblast cells; and
(d) about four weeks after (c) or about eight weeks after (c), providing a fourth dose of fibroblasts comprising about 100 million fibroblast cells.

10. The method of any of claims 1-9, wherein the subject has cholestatic liver disease.

11. The method of any of claims 1-10, wherein the subject has inflammatory bowel disease (IBD).

12. The method of any of claims 1-10, wherein the subject does not have IBD.

13. The method of any of claims 1-12, wherein the subject has elevated alkaline phosphatase levels prior to providing the fibroblasts or derivatives thereof.

14. The method of any of claims 1-13, wherein, subsequent to the providing, the subject shows an improvement of a 5-D itch score, an Amsterdam cholestatic complaints score, and/or a liver stiffness transient elastography score.

15. The method of any of claims 1-14, wherein the fibroblasts or derivatives thereof do not cause an adverse event in the subject, wherein the adverse event is hepatoxicity, progressive multifocal leukoencephalopathy, cholangiocarcinoma, one or more complications due to portal hypertension, leucopenia, lymphopenia, colorectal cancer, infusion-related reactions, infection, acute respiratory failure, acute respiratory distress syndrome, Torsade de pointer, ventricular fibrillation, ventricular tachycardia, malignant hypertension, convulsive seizure, agranulocytosis, aplastic anemia, toxic epidermal necrolysis, Stevens-Johnson syndrome, hepatic necrosis, acute liver failure, anaphylactic shock, acute renal failure, pulmonary hypertension, pulmonary fibrosis, confirmed or suspected endotoxin shock, confirmed or suspected transmission of infectious agent by a medicinal product, neuroleptic malignant syndrome, malignant hyperthermia, spontaneous abortion, stillbirth, and/or fetal death.

16. The method of any of claims 1-15, wherein the fibroblasts are allogenic fibroblasts, autologous fibroblasts, or xenogenic fibroblasts.

17. The method of any of claims 1-16, wherein the fibroblasts are derived from placenta, cord blood, peripheral blood, omentum, hair follicle, skin, bone marrow, adipose tissue, endometrium, or Wharton's Jelly.

18. The method of any of claims 1-17, further comprising providing to the subject one or more additional agents, wherein the additional agent comprises n-acetylcysteine, ascorbic acid, alpha lipoic acid, human chorionic gonadotropin, VEGF, TNF-α, retinoic acid, alpha tocopherol, interleukin-3, G-CSF, GM-CSF, leukemia inhibitory factor, placental growth factor, angiopoietin, hydrogenated water, and/or NGF.

19. The method of any of claims 1-18, further comprising providing to the subject an additional cell therapy, wherein the additional cell therapy is capable of suppressing liver inflammation in the subject.

20. The method of claim 19, wherein the additional cell therapy comprises natural killer T (NKT) cells.

21. The method of claim 20, wherein the NKT cells are activated with alpha galactosylceramide prior to providing the additional cell therapy to the subject.

22. The method of claim 19, wherein the additional cell therapy comprises immature dendritic cells.

23. The method of claim 22, wherein the immature dendritic cells produce more than 50 ng of interleukin-10 per 10,000,000 cells.

24. The method of claim 22 or 23, wherein the immature dendritic cells do not express HLA II.

25. The method of any of claims 1-24, further comprising providing to the subject an effective amount of endothelial progenitor cells.

26. The method of claim 25, wherein the endothelial progenitor cells are derived from the subject.

27. The method of claim 25 or 26, further comprising mobilizing the endothelial progenitor cells in the subject.

28. The method of claim 27, wherein mobilizing the endothelial progenitor cells comprises administration of granulocyte colony stimulating factor (G-CSF) to the subject.

29. The method of claim 27, wherein mobilizing the endothelial progenitor cells comprises administration of an effective amount of granulocyte macrophage colony stimulating factor (GM-CSF) to the subject.

30. The method of claim 27, wherein mobilizing the endothelial progenitor cells comprises administration of an effective amount of interleukin (IL)-3 to the subject.

31. The method of claim 27, wherein mobilizing the endothelial progenitor cells comprises administration of an effective amount of thrombopoietin (TPO) to the subject.

32. The method of claim 27, wherein mobilizing the endothelial progenitor cells comprises administration of an effective amount of FLT3 ligand (FL) to the subject.

33. The method of any of claims 25-32, wherein the endothelial progenitor cells are allogenic.

34. The method of any of claims 25-33, wherein the endothelial progenitor cells are derived from placenta, cord blood, peripheral blood, omentum, hair follicle, adipose derived stromal vascular fraction, skin, bone marrow, adipose tissue, endometrium, Wharton's Jelly, or a combination thereof.

35. The method of any of claims 1-34, further comprising providing an effective amount of regenerative cells to the subject.

36. The method of claim 35, wherein the regenerative cell is a stem cell.

37. The method of claim 36, wherein the stem cell is a hematopoietic stem cell.

38. The method of claim 37, wherein the hematopoietic stem cell expresses CD34, CD133, c-kit, and/or thrombopoietin receptor.

39. The method of claim 37 or 38, wherein the hematopoietic stem cell does not express CD38.

40. The method of any of claims 37-39, wherein the hematopoietic stem cell is an autologous hematopoietic stem cell.

41. The method of any of claims 37-39, wherein the hematopoietic stem cell is an allogenic hematopoietic stem cell.

42. The method of any of claims 37-39, wherein the hematopoietic stem cell is a xenogenic hematopoietic stem cell.

43. The method of any of claims 37-42, wherein the hematopoietic stem cell is derived from adipose, bone marrow, peripheral blood, mobilized peripheral blood, cord blood, or a mixture thereof.

44. The method of any of claims 1-43, further comprising providing to the subject an effective amount of mesenchymal stem cells.

45. The method of claim 44, wherein the mesenchymal stem cell expresses CD90, CD105, and/or CD73.

46. The method of claim 44 or 45, wherein the mesenchymal stem cell does not express HLA, CD34, and/or CD14.

47. The method of any of claims 44-46, wherein the mesenchymal stem cell is plastic adherent.

48. The method of any of claims 44-47, wherein the mesenchymal stem cell is allogenic to the subject.

49. The method of any of claims 44-47, wherein the mesenchymal stem cell is autologous to the subject.

50. The method of any of claims 44-49, wherein the mesenchymal stem cell is derived from adipose, bone marrow, peripheral blood, mobilized peripheral blood, menstrual blood, fallopian tube, or cord blood.

51. The method of any of claims 1-50, further comprising providing to the subject an effective amount of exosomes derived from one or more stem cells, wherein the one or more stem cells comprise hematopoietic stem cells, mesenchymal stem cells, or a combination thereof.

52. The method of claim 51, wherein the exosomes are derived from the one or more stem cells via ultracentrifugation.

53. The method of claim 51, wherein the exosomes are derived from the one or more stem cells via chromatography.

54. The method of claim 51, wherein the exosomes are derived from the one or more stem cells via affinity purification.

55. The method of any of claims 51-54, wherein an outer surface of the exosomes comprises phosphatidylserine, CD9, CD19, and/or a tetraspanin protein.

56. The method of any of claims 51-55, further comprising stimulating the one or more stem cells to secrete the exosomes.

57. The method of claim 56, wherein the stimulating comprises culturing the one or more stem cells in hypoxic conditions.

58. The method of claim 57, wherein the hypoxic conditions comprise between 0.01% and 10% oxygen.

59. The method of claim 58, wherein the hypoxic conditions comprise 3% oxygen.

60. The method of any of claims 56-58, wherein the one or more stem cells are cultured in the hypoxic conditions for less than 14 days.

61. The method of claim 60, wherein the one or more stem cells are cultured in the hypoxic conditions for about 4 days.

62. The method of any of claims 1-61, further comprising culturing the fibroblasts with one or more agents prior to providing the fibroblasts or derivatives thereof to the subject.

63. The method of claim 62, wherein the agent is metformin.

64. The method of claim 62, wherein the agent is oxytocin.

65. The method of claim 62, wherein the agent is chorionic gonadotropin.

66. The method of any of claims 62-65, wherein the agent is capable of enhancing production of an angiogenic cytokine in the fibroblasts.

67. The method of claim 66, wherein the angiogenic cytokine is VEGF.

68. The method of claim 66, wherein the angiogenic cytokine is FGF-1.

69. The method of claim 66, wherein the angiogenic cytokine is FGF-2.

70. The method of claim 66, wherein the angiogenic cytokine is IGF-1.
